(19) United States
(12) Patent Application Publication
(10) Pub. No.: US 2023/0351263 A1
(43) Pub. Date: Nov. 2, 2023

(54) ACTIVE MACHINE LEARNING MODEL FOR TARGETED MASS SPECTROMETRY DATA ANALYSIS

(71) Applicant: The Administrators of the Tulane Educational Fund, New Orleans, LA (US)

(72) Inventors: Duran BAO, New Orleans, LA (US); Jia FAN, New Orleans, LA (US); Ye Tony HU, New Orleans, LA (US); Qingbo SHU, New Orleans, LA (US); Bo NING, New Orleans, LA (US); Christopher J. LYON, New Orleans, LA (US)

(21) Appl. No.: 18/302,729

(22) Filed: Apr. 18, 2023

Related U.S. Application Data
(60) Provisional application No. 63/332,065, filed on Apr. 18, 2022.
(60) Provisional application No. 63/449,437, filed on Mar. 2, 2023.

(51) Int. Cl. G06N 20/00; G06N 5/022

(52) U.S. Cl. CPC G06N 20/00; G06N 5/022

(57) ABSTRACT

This disclosure describes a method and system for active machine learning model that automatically and continuously improve the model with high accuracy, sensitivity, specificity and universality.

### PRIOR RELATED APPLICATIONS

This invention claims priority to U.S. App. Ser. No. 63/332,065, filed on Apr. 18, 2022, and Ser. No. 63/449,437, filed on Mar. 2, 2023, each of which is incorporated by reference in its entirety herein for all purposes.

### FEDERALLY SPONSORED RESEARCH STATEMENT

Not applicable.

### FIELD OF THE DISCLOSURE

The disclosure generally relates to methods of identifying samples with a biomarker using mass spectrometry, and more specifically to the construction of active learning method an generation of training sample sets for machine learning applications on a variety of samples with reduced human operation.

### BACKGROUND OF THE DISCLOSURE

Liquid chromatography with tandem mass spectrometry (LC-MS/MS) has become a promising analytical technique for biomarker identification and quantification in disease diagnosis. However, its clinical application is limited by the fact that the mass spectrometry (MS) data output must be analyzed by technicians with extensive MS experience.

LC-MS/MS can be applied to both qualitative and quantitative analysis in many fields, such as microbiology, toxicology, and clinical diagnosis. LC-MS/MS analysis can be utilized to identify prognostic biomarkers. One example of such a use case is with CFP-10, a 10-kDa biomarker present in active TB infections. CFP-10 is tested for in rapid and high-sensitivity quantification of blood-based assays. Through LC-MS/MS technology, samples containing this active Mtb-specific peptide are more easily and rapidly identified, compared to culture tests which can be more time-consuming. CFP-10 and other peptides have been authorized as a diagnostic standard by the FDA, and uses of additional biomarkers are pending clinical trials. However, there are many challenges in utilizing LC-MS/MS technology in clinical diagnosis. First, long-term training is necessary to enable technicians to read and analyze data. Second, there is an issue of operator subjectivity when the target peptide signals are ambiguous. Third, with recent increases in the amount of data generation, manual analysis of spectrometry data may not be able to keep up with data growth.

Machine learning (ML) methods have been introduced into LC-MS/MS data analysis. Due to the potential for thousands of proteins and complex mixtures within a single sample, downstream analysis increasingly utilizes both supervised and unsupervised ML methods. Despite advances in these ML methods, there are still many challenges in using ML for LC-MS/MS data analysis. First, machine learning methods often have high performance within internal datasets but low performance in external independent ones, typically due to variances in studies, patients and mass spectrometry devices. Second, pipelines for predictions are difficult to build for limited sample sizes. Third, many ML methods are often considered “black boxes,” and researchers and physicians will not accept clinical predictions without proper rationale and justifications. Forth, in conventional ML methods, models couldn't be built until a large accumulation of samples was available or new samples were manually injected into the model as candidates.

Therefore, there is the need for a machine learning method that has high performance with both internal and external datasets, that can be built using limited sample sizes, that has proper rationale and justifications for the prediction results, and that can be automatically improved without having to manually input datapoints into the model.

### SUMMARY OF THE DISCLOSURE

To address the challenges encountered in machine learning as applied to mass spectrometry, a novel method is described herein to efficiently and effectively build prediction models for mass spectrometry, especially with regards to infectious diseases such as tuberculosis, HIV, COVID-19, etc. The machine learning method described herein is used for building a database for identifying a biomarker in a sample using mass spectrometry (MS), and the method comprises: (a) obtaining mass spectrometry (MS) data from a sample, (b) extracting, by a computer, features from the MS data; (c) inputting, by a computer, the features extracted in step b) into a trained prediction model, wherein the prediction model is trained to predict presence of an analyte in said sample; and (d) generating an output, wherein the output comprises prediction of the presence of the analyte in said sample.

In another aspect of this disclosure, a method of building a machine learning pipeline is described. The method comprises the steps of: a) extracting features from mass spectrometry (MS) or liquid-chromatography mass spectrometry (LC-MS) data regarding presence of an analyte; b) constructing, by one or more computing devices that implement a machine learning program, two or more machine learning models using an active learning workflow; c) optimizing, by the one or more computing devices, the machine learning model; and d) selecting, by the one or more computing devices, a best model; wherein the features in step a) comprises statistical and morphological features.

In another aspect of this disclosure, a system is described. The system comprises: at least one processor; a memory, storing program instructions that when executed by the at least one processor causes the at least one processor to perform a machine learning pipeline, the machine learning pipeline is configured to perform at least one of the following modes: i) training mode: (A) receive mass spectrometry data of a sample; (B) extract at least one feature from the mass spectrometry data, wherein the at least one feature is a statistical feature and/or a morphological feature; (C) optimize training dataset by active learning strategy; and (D) select a best prediction model; ii) prediction mode: prediction mode: (A) receive mass spectrometry data of a sample; (B) extract at least one feature from the mass spectrometry data, wherein the at least one feature is a statistical feature and/or a morphological feature; and (C) generate an output of determining whether an analyte is present in the sample.

In another aspect of this disclosure, one or more non-transitory, computer-readable storage media is described. The non-transitory, computer-readable storage media stores program instruction that when executed on or across one or more computing devices cause the one or more computing devices to implement at least one of: i) training mode: (A) receive mass spectrometry data of a sample; (B) extract at least one feature from the mass spectrometry data, wherein the at least one feature is a statistical feature and/or a morphological feature; (C) optimize training dataset by active learning strategy; and (D) select a best prediction model; ii) prediction mode: prediction mode: (A) receive mass spectrometry data of a sample; (B) extract at least one feature from the mass spectrometry data, wherein the at least one feature is a statistical feature and/or a morphological feature; and (C) generate an output of determining whether an analyte is present in the sample.

According to any method, system or non-transitory computer-readable storage media described herein, the features comprise statistical features and morphological features.

According to any method, system or non-transitory computer-readable storage media described herein, the statistical features comprise Peak_Max, Peak_Area, Peak_Ratio, and/or Peak_Shift.

According to any method, system or non-transitory computer-readable storage media described herein, morphological features comprise: updown-difference, similarity, jaggedness, modality, symmetry, and/or FWHM.

According to any method, system or non-transitory computer-readable storage media described herein, the morphological features are extracted using normalized MS data.

According to any method or system described herein, the morphological features are extracted using normalized MS data.

According to any method, system or non-transitory computer-readable storage media described herein, the output further comprises feature importance.

According to any method, system or non-transitory computer-readable storage media described herein, the feature importance is obtained by calculating a Shapley Additive exPlanation (SHAP) value for each extracted feature, and sorting the features by the SHAP value.

According to any method described herein, the active learning workflow comprises at least one of: (i) label balancing, and (ii) even score distribution.

According to any method described herein, the even score distribution evaluates at least one of the following: accuracy, sensitivity, specificity, area under curve (AUC), and F1.

As used herein, “PeakMax” refers to the target maximum intensity between the internal standard peak boundaries. The calculation of PeakMax is expressed as Max {T1, T2, . . . , Tn-1, Tn}.

As used herein, “PeakArea” refers to the summation of all target intensities between the internal standard peak boundaries. The calculation of PeakArea is expressed as Σᵢ₌₁ⁿTn.

As used herein, “PeakRatio” refers to the ratio among the internal standard peak boundaries of summation of target intensities to summation of internal standard intensities. The calculation of PeakRatio is expressed as Σᵢ₌₁ⁿTn/Σᵢ₌₁ⁿSn.

As used herein, “Peak_shift” refers to the retention time difference between internal standard and target intensities. The calculation of Peak_shift is expressed as Rm-R(Tmax).

As used herein, “Updown-Dif” refers to the sift in target peak, which is defined as the difference intensities between the left and right peak boundaries. The calculation of Updown-Dif is expressed as |Norm (Tmax)−Norm (Smax)|.

As used herein, “Similarity” refers to the Pearson correlation coefficient between the internal standard and target intensities. The calculation of Similarity is expressed as Corr ({T1, T2, . . . Tn}, {S1, S2, . . . , Sn}).

As used herein, “Symmetry” refers to the Pearson correlation coefficient between the left and right halves of the peak intensity vector divided at the timepoint in the middle. The calculation of Symmetry is expressed as Corr ({T1, T2, . . . Tmax}, {Tn Tn-1, Tmax}).

As used herein, “Jaggedness” refers to the difference of mean intensities at the same timepoint between standard and target peaks. The calculation of Jaggedness is expressed as

$$\frac{1}{n} {\sum }_{i = 1}^{n} ( Norm ( T ) - Norm ( S ) ) .$$

As used herein, “Modality” refers to the largest unexpected dip in the peak, normalized by peak height. The calculation of Modality is expressed as:

|First (DPOS)−LAST(DPOS)|

wherein

Di=Norm(Ti)−Norm(Si)

DPOS={. . . ,Di, . . . }, (Di≥0)

As used herein, “FWHM” refers to the retention time scans between two half-maximum intensities. The calculation of Modality is expressed as

|Rleft−Rright|

wherein

Rleft=R(Tmax/2), (R<Rm)

Rright=R(Tmax/2), (R>Rm)

As used herein, “machine learning pipeline” refers to the end-to-end construct that orchestrates the flow of data into, and output from, a machine learning model (or set of multiple models). It includes raw data input, features, outputs, the machine learning model and model parameters, and prediction outputs.

As used herein, “label balancing” refers to selecting LC-MS/MS raw data based on positivity rate of the target analyte.

As used herein, “even score distribution” refers to

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| AL | Active learning |
| AUC | Area under curve |
| LC-MS | Liquid chromatography-mass spectrometry |
| ML | Machine learning |
| MS | Mass spectrometry |
| ROC | Receiver operating characteristics |
| SHAP | Shapley Additive exPlanations |

### BRIEF DESCRIPTION OF THE DRAWINGS

FIG. 1A shows construction of dynamic machine learning pipeline. a. LC-MS/MS data input and processing. Peak boundaries help with processing the internal standard and target peptide signals; b. Feature extraction. Engineering features were extracted by corresponding formula and were grouped by statistical and morphological categories. c. Machine learning algorithms were used to build analysis pipeline. d. Model performance were evaluated by internal validation. d. Extension of pipeline application. This included small training set validation, external dataset validation, and explanation of model. f. Dynamic active learning pipeline. It enabled the model to be optimized by new samples automatically.

FIG. 1B shows the flow chart of the machine learning pipeline of this disclosure.

FIG. 2 shows comparison of model performance built by traditional and novel features. a. Five metrics: accuracy, sensitivity, specificity, AUC, F1 scores were displayed model performance. Logistic regression (left), random forest (middle), gradient boosting (right) algorithms were used for model building. Orange and purple dots mean models built by traditional and novel features separately. Training datasets were resampled by 5 times. b. ROC curves of model evaluations. The curves and AUC values were grouped by algorithm: logistic regression (left, AUC values from traditional features: 0.5636±0.04 and from novel features: 0.9715±0.0312), random forest (middle, AUC values from traditional features: 0.8123±0.0215 and from novel features: 0.9756±0.0172), gradient boosting (right, AUC values from traditional features: 0.8024±0.0547 and from novel features: 0.9275±0.03 5).

FIG. 3 shows best algorithm to match defined features. a. Schematic diagram for different training subsets selected from MLIT_TR randomly, the same testing dataset were evaluated by all the models, the whole model bunching and evaluation were repeated five times by different training sets; b. Performance of three algorithms in different training size datasets. Three algorithms were displayed separately. The trendlines were established among the AUC values with the increasing training size. c. Comparison of three algorithms performance in the unstable phase. d. Comparison of three algorithms performance in the stable phase. (****: p<0.0001, ns: no significance.)

FIG. 4 shows performance in independent LC-MS/MS datasets. a. Schematic diagram for comparing machine learning algorithms by two independent datasets with model bunching. b. AUC values were collected by algorithms in INDTS1 datasets. c. Pairwise comparison of AUC values from model bunching. (****: p<0.0001) d. AUC values were collected by algorithms in INDTS2 datasets. e. Pairwise comparison of AUC values from model bunching. (****: p<0.0001).

FIG. 5 shows active learning strategy on training data optimization. a. Scheme of the active learning optimization procedures. b. Comparison of balancing and unbalancing training samples effects on model performance. c. External validation (x axis means the training samples resource, all model evaluations were tested by Italy cohort dataset. Purple dots means that models built by all of the training samples and red dots means that by active learning selecting samples.)

FIG. 6A illustrates the exemplary statistical features extracted in this disclosure.

FIG. 6B illustrates the exemplary morphological features extracted in this disclosure.

FIG. 6C shows feature importance analysis. a. The best model selected from Set 1. Schematic diagram for features, category, and grid meaning in mosaic plot. SHAP value of each sample and feature were shown in the scatter plot. Top 15 features of absolute SHAP value mean rankings were displayed. b. Comparison of two categories of features by SHAP values. Numbers on the dots were shown as SHAP value rankings. c. The distribution of two categories of features in five training sets. Red: Morphological; Blue: Statistical. Wilcoxon signed rank sum Test in two categories of features. d. Comparison of integrated and transitional features by SHAP values. In morphological category, SHAP values of integrated (0.0146±0.0165) and transitional (0.0109±0.0125); in statistical category, SHAP values of integrated (0.0041±0.0065) and transitional (0.002±0.0028) e. Comparison of small and large transitional features by SHAP values. In morphological category, SHAP values of small transitional (0.0046±0.006) and large transitional (0.0109±0.0125); in statistical category, SHAP values of integrated (0.0052±0.0089) and transitional (0.002±0.0028).

FIG. 7 shows LC-MS/MS data visualization. Left lane: Internal standard intensity, right lane: target peptide intensity; Upper row: Sample of ‘MLIT0003’ (label was CFP-10 positive), Lower row: Sample of ‘MLIT0005’ (label was CFP-10 negative). X-axis: Retention time (RT), y-axis: mass spectrometry data intensity. The highlighted zone was peak boundaries defined by illustration.

FIG. 8 shows a comparison among models built by traditional and novel features. Paired-t-test was used as statistical analysis. (ns: not significant; ****: p value <0.0001)

FIG. 9 shows ROC curves from models by four more re-samplings. Upper row was the model performance built by training datasets. Lower row was the model performance built by testing dataset.

FIG. 10 shows comparison between ‘unstable’ and ‘stable’ model phases.

FIG. 11 shows SHAP values from models built from MLIT-TR2 to MLIT-TR5.

FIG. 12 shows analysis of ‘unstable’ and ‘stable’ algorithm performance for logistic regression (LR), random forest (RF), and gradient boosting (GB) algorithms.

### DETAILED DESCRIPTION

Detailed descriptions of one or more preferred embodiments are provided herein.

It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

First, the differences in LC-MS/MS data between positive and negative TB samples were observed and features were engineered which contained both statistical (based on the original chromatography data) and morphological (based on normalized data) aspects from chromatography (rather than importing raw data directly into the models). The calculations were based on the target peptide intensity, which was identified by the internal standard (IS) concentrated intensity. Second, three ML algorithms were utilized: logistic regression (LR), random forest (RF), and gradient boosting (GB), which were specialized in binary label classification. Additionally, to fix certain practical issues, such as limited and biased-label samples, an active learning method, which helped to optimize training samples, was introduced to solve them from different aspects. This increased the training dataset to avoid overfitting or unbalanced sensitivity and specificity in the models. This dynamic analysis method also improved the pipeline to make predictions efficiently and rapidly. Finally, we assessed the importance of individual features on model performance through Shapley Additive explanations (SHAP) values.

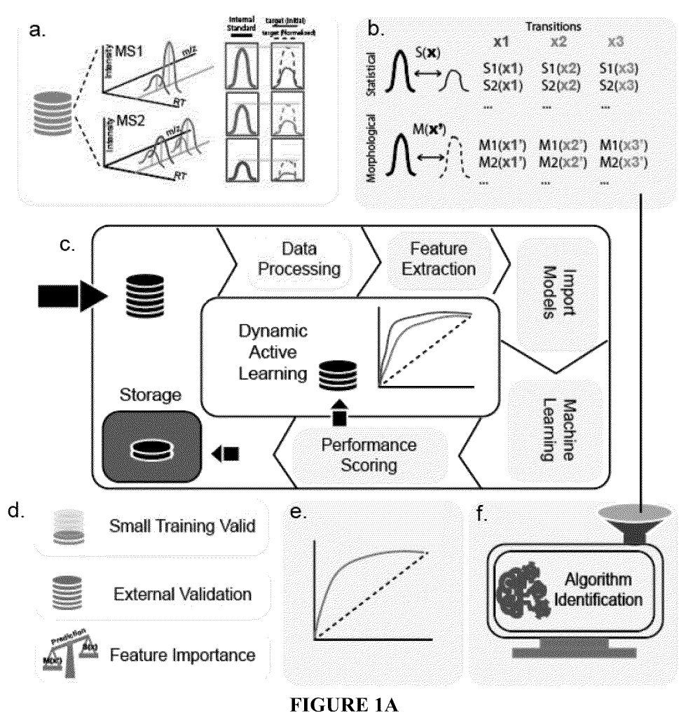

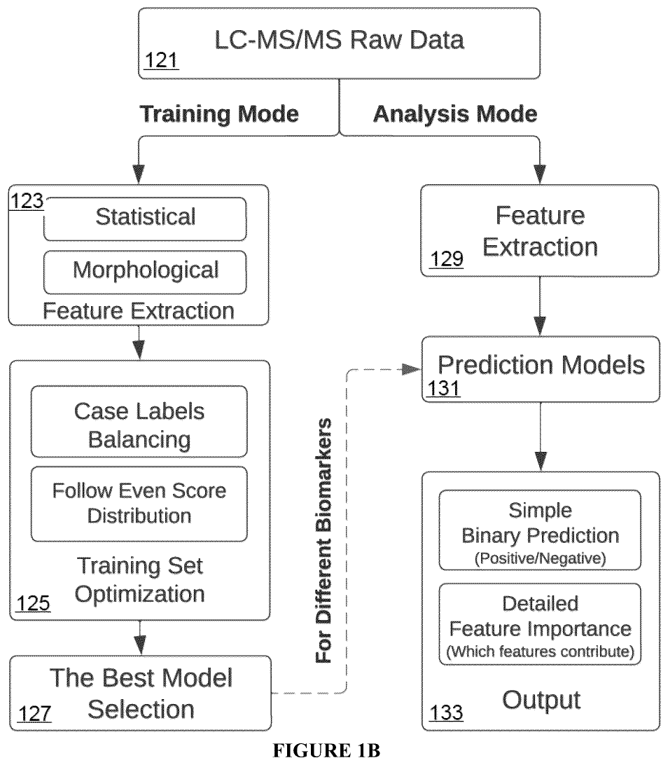

In one embodiment of this disclosure, a method of building a self-improving machine learning (ML) pipeline is described with reference to FIG. 1B. In step 121, LC-MS/MS raw data of a sample that may contain an analyte of interest is obtained through ordinary means. The LC-MS/MS raw data typically includes a mass spectrum, a plot of intensity as a function of the mass-to-charge ratio. A mass spectrum is a type of plot of the ion signal as a function of the mass-to-charge ratio. These spectra are used to determine the elemental or isotopic signature of a sample, the masses of particles and of molecules, and to elucidate the chemical identity or structure of molecules and other chemical compounds.

Depending on the need, an operating personnel will direct the program to perform either the training mode or the analysis mode. The training mode will employ different algorithms to classify and label the MS raw data, in order to train the ML pipeline. The analysis mode will analyze the raw data based on the trained ML pipeline and make a prediction of whether the analyte is present in the sample.

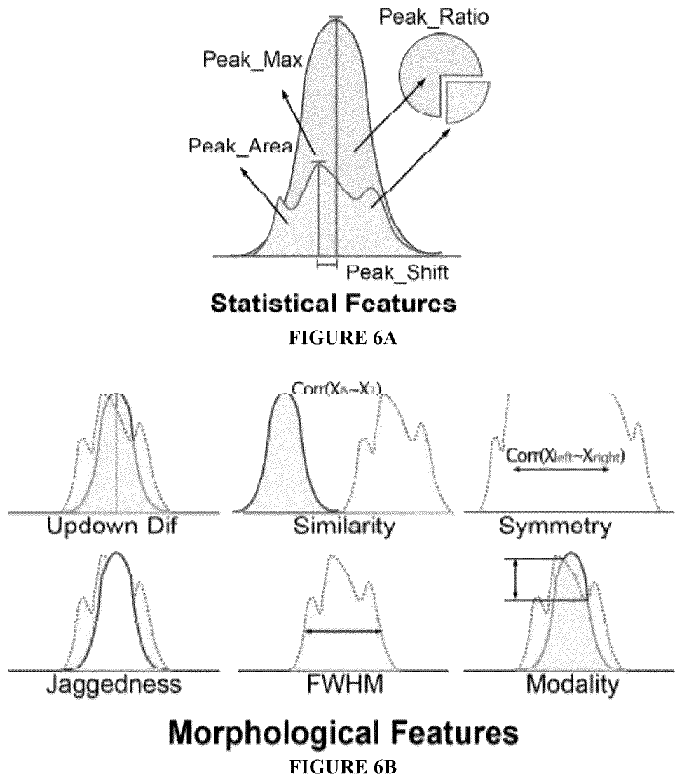

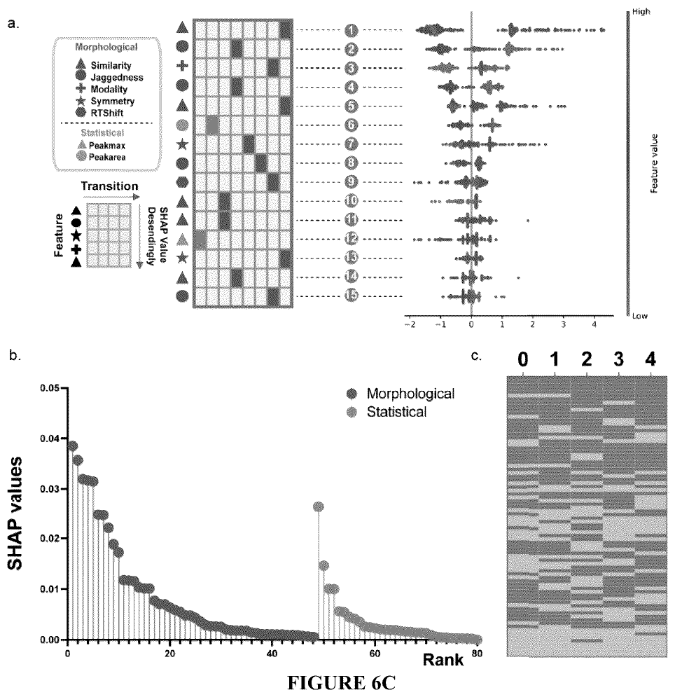

In the training mode, the first step is to perform feature extraction 123. Conventionally, the analysis of the MS raw data focuses on the values of the raw data. The inventors of this disclosure discovered that certain features may offer better efficiency in identifying the target peptide. Therefore, after receiving the raw LC-MS/MS data, it is important to extract these features. As discussed herein, the features are divided into statistical features (FIG. 6A) and morphological features (FIG. 6B). While the statistical features are extracted following internal standard peptide peak boundaries, some features describing the shape of chromatography are also extracted to mimic the human vision. The morphological features offer the relationship between target and internal standard, which are considered in manual operation. The morphological features are extracted after normalization to dismiss the variance of samples. The equations for extracting statistical and morphological features are listed in Table 1.

In step 125, after the features are extracted, the training dataset is constructed and optimized by first balancing the case labels such that the dataset could assist the model to cover all characteristics to make unbiased predictions. Specifically, in the active learning (AL) method for dynamic training dataset construction, the randomly selected positive sample rate is a component of the training dataset.

The training dataset is also constructed and continuously optimized by implementing an active learning strategy.

In step 127, a plurality of machine learning algorithms are used for modeling, and the results from each algorithm are compared by following a scoring system in order to select the model with the best prediction result. Three algorithms are chosen as examples (logistic regression, random forest, gradient boosting), but other machine learning algorithms can also be used. The scoring system evaluates each algorithm for accuracy, sensitivity, specificity and F1 scores, as well as the AUC (area under curve) values of receiver operating characteristics (ROC). The algorithm with the greatest predictive power is again tested with independent datasets to evaluate the universality thereof, as well as testing the performance of different sample sizes.

Once the training mode is completed, the ML pipeline can then switch to analysis mode for determining if an analyte is present in a sample. Or alternatively, the ML pipeline can be used in a different target biomarker to test its performance.

In step 129, the feature extraction step is similar to that of step 123.

In step 131, the algorithm with the best predictive power and universality selected in step 127 is used to analyze and predict the presence of target analyte in the sample.

In step 133, the ML pipeline generates an output. It is noted that in addition to simple binary prediction (as in positive/negative), the ML pipeline can further calculate SHAP (Shapley Additive exPlanations) value to each of the features. SHAP values are based on additive feature attribution method, and can provide further insight into machine learning algorithms. By calculating the SHAP value of each feature, the pipeline can then rank the importance of features, which could contribute to future training.

Methods

Sample Collection and Experiment Procedure.

Three datasets, Machine Learning Internal Training (MLIT), Independent Dataset 1 (INDTS1), Independent Dataset 2 (INDTS2), and Independent Dataset 3 (INDTS3) were used. Each dataset was acquired by Thermo Altis triple quadrupole mass spectrometry coupled with Ultimate 3000 nano-liquid chromatography. Data were labeled as either targeted peptide “positive” or “negative” which were determined based on manual analysis by reviewing the raw spectrum, extracting the dotp and rdotp provided by Skyline program, and calculating SNR by the Freestyle program.

The MLIT dataset was used to train and test ML algorithms, and served as the control dataset. There are 287 samples in the MLIT dataset, with 159 “positive” samples and 128 “negative” samples. Three additional independent datasets, INDTS1, INDTS2, and INDTS3, were used for external validation and predicted performance of the best ML model and model's reproducibility and generalizability. The INDTS1 dataset contained 201 samples, with 28 (#%) labeled as “positive” and 173 labeled as “negative.” The INDTS2 dataset contained 36 samples, with 31 labeled as “positive” and 36 labeled as “negative.” The INDTS3 dataset contained 98 samples, with 19 labeled as “positive” and 79 labeled as “negative.”

Data visualization.

LC-MS/MS chromatography data was visualized using html file formats, and visualizations included transition peaks, m/z, and label information. Several chromatography samples were compared visually and differences between “positive” and “negative” samples were noted. Different colors were utilized to present transitions, and the final curves were smoothed and compressed to fit in one window.

Data Processing.

To import raw data into Python 3.7, ProteoWizard software (http://proteowizard.sourceforge.net/) and the pyMZML 2.4.7 package were utilized to convert MZML files to readable Pickle files and into numeric formats that could be used by the Python ‘Numpy’ and ‘Pandas’ packages. Peak boundaries through retention time were computed using a 95% confidence interval of the internal standard maximum peak area. Integrated and transitional targeted signals were identified from MS1 and MS2 data within these peak boundaries.

Features Extraction.

Targeted peptide absolute intensities were extracted from the original data. The seven transition ions of the target peptide were at 622.29 m/z, 693.33 m/z, 822.37 m/z, 950.43 m/z, 1021.47 m/z, 1134.55 m/z, and 1235.60 m/z, and the seven transition ions for the internal standard were all with+10 m/z shift compared to the transition ions of targeted peptide. These were captured at the second stage of mass spectrometry (MS2) which generated the ion fragment spectrums. The methodology used to collect chromatography features were initially developed from an R package, and partial features were selected and modified to meet our study's objectives. We defined the features as statistical and morphological features based on whether their resource came from original target data or the parameters calculated by peak shapes. Examples of statistical features include: Peak_Max, Peak_Cum, Peak_Ratio, and Peak_Shift.; they described the target peptide signals directly. We previously identified 1021.47 m/z as the highest transition ion. Therefore, we normalized each transition intensity by this transition to dismiss the effects among different batches and studies. Also, the integrated intensity derived from the summation of these processed intensities. The morphological features were calculated based on normalization of the targeted data by corresponding internal standards. The seven transition peaks of the target peptide compared to internal standards were identified at 632.29 m/z, 703.33 m/z, 832.37 m/z, 960.43 m/z, 1031.47 m/z, 1144.55 m/z, 1245.60 m/z. Examples of morphological features include: Updown-Dif, Similarity, Symmetry, Jaggedness, Modality and FWHM.

When no signals were present at certain transitions or if there was no obvious target peptide signal, the intensity was labeled as ‘0’ or ‘negative’ at the corresponding retention time. This was done to reduce any potential misinterpretation by the ML algorithms.

In total, 80 features were calculated and engineered to detect if the samples contained the target peptide CFP-10, and all features were utilized in ML algorithms. A complete list of feature names, descriptions, and calculation equations are provided in Table 1.

Machine Learning Algorithms.

Three machine learning algorithms—logistic regression, random forest, and gradient boosting—were used for modeling, and the classification pipeline was implemented through the scikit-learn-0.23 package in Python 3.7. For each algorithm, hyperparameters were adjusted to improve predictive powers, and the models with the highest accuracy were selected to conduct further analysis and testing. For logistic regression algorithms, the hyperparameters penalty and max_iter were adjusted. For random forest algorithms, the hyperparameters max_features, n_estimators, and max_depth were adjusted. For gradient boosting algorithms, the hyperparameters n_estimators, max_depth, max_features, and learning_rate were adjusted. Models were optimized by smooth hyperparameter adjustments and K-Fold cross validation (In this study, K=10) in the training dataset. These pipelines were not only used to classify samples, but also to observe which algorithm performed best in both internal and external datasets.

Algorithm Performance Evaluation.

The MLIT dataset was utilized to train and test the aforementioned machine learning models, while INDTS1, INDTS2 and INDTS3, were used to determine the ML models' universality. The MLIT dataset was divided into a training subset (MLIT-TR; 80% of data points, or 230 samples) and a testing subset (MLIT-TS; 20% of data points, or 57 samples). This train/test data division was conducted five times using pseudo random seeds, and training sets were labeled as MLIT-TR1 through MLIT-TR5.

Additionally, in the training model procedures, a 10-fold cross validation method was utilized, in which data was randomly and evenly assigned to one of 10 subsets. These subsets were named MLIT-TR1-1 (N=23), MLIT-TR1-2 (N=46), . . . , MLIT-TR1-10 (N=230), and the training subsets were used to explore the impact of sample size on model performance. Every subset was tested using the nine other subsets to optimize parameters within each machine learning model. The general performance metrics used to evaluate the models included accuracy, sensitivity, specificity and F1 scores. In addition, the AUC values of receiver operating characteristics (ROC) were calculated to compare the predictive power of the models.

The models with the greatest predictive power were later tested with the independent datasets INDTS1, INDTS2, and INDTS3 to analyze the universality of the models, and to observe if smaller sample sizes would impact model performance using independent datasets. SHAP Value for Feature Importance.

SHAP values, which are based on additive feature attribution methods, can provide further insight into machine learning algorithms. Using the Python package “SHAP-0.37.0”, SHAP values were calculated for algorithms in the present study. The resulting SHAP values could be positive or negative, which indicate increasing to event or decreasing to un-event. SHAP values across the 80 features were compared to understand which features had the strongest effects on classification. Feature importance was ranked by mean of absolute SHAP values, with higher ranking features indicating a higher level of contribution to the predictive capabilities of the model.

Statistical Analysis.

Five performance metrics were analyzed in the ML algorithms whenever a pseudo random seed or parameter of the algorithm was changed. Two phases, unstable and stable performance, were grouped by one-way ANOVA with Bonferroni adjustment (SAS 9.4, p <0.05). The difference among three algorithms were tested by pairwise-t test (SAS 9.4, p <0.05). SHAP values were calculated through Python 3.7, SHAP-0.37.0 Package. Nonparametric comparison was tested through Wilcoxon Signed Rank Sum Test (SAS 9.4, p<0.05). Line plots and bar plots were generated by Graphpad Prism 9.0.

Active Learning Workflow

To test active learning optimization's performance, two datasets, training (80%) and testing (20%) datasets, were used for the validation. First, we calculated the scores of each sample in the training dataset by the best algorithm of the three (random forest in this example), and we sorted the samples by the scores. Second, we applied a systematic sampling method to identify an initial dataset (10 negative and 10 positive out of training dataset separately), and the remaining samples were assigned to the dynamic dataset which mimicked incoming samples as an external dataset. Third, using the initial dataset to re-build the model again, we then calculated the new scores of each sample in the dynamic samples. Fourth, the training sample pool would dismiss the samples which scores at the range of p (0<p<0.5; we used 0.45) and 1-p. Finally, if the positive and negative samples were not balanced, we used systematic sampling methods to select the samples from the exceeding labeled side. We imported the new balanced dataset to build the model again and gained the optimized training model.

Results

Data Conversion, Visualization, and Feature Extraction.

All raw data was acquired by Thermo TSQ Altis Devices. To import the MS data into our customized analyzed pipeline, we converted the data to ‘mzML’ format which could be recognized by the Python environment without information loss. To enable the pipeline to use the precise data of CFP-10, the target peptide signals were extracted following internal standard peptide peak boundaries and the rest of MS data was discarded.

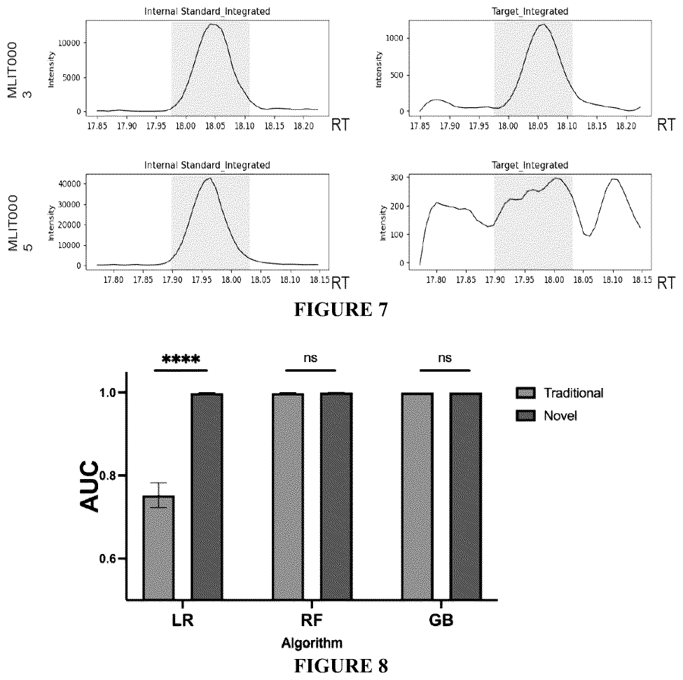

To regard the MS data at the range of selected boundaries as comprehensive information to describe the peptide, we extracted some parameters, four statistical features, from the initial data instead of raw intensity of each retention time scan. These features could replace more repeatable and meaningless information efficiently. Additionally, to mimic the human vision in manual analysis, some features describing the shape of chromatography were also extracted and defined as morphological features. This category of features offers the relationship between target and internal standard by ‘Similarity’, the quality of the peak by ‘Symmetry’, and many other parameters which were considered in manual operation. To enrich the characteristics of the target peptide, we extracted features from not only MS1 but also MS2 fragment intensities. The CFP-10 peptide is fragmented into 7 transitions, with the 1021.47 m/z as the highest. To dismiss the variance of samples from different populations or studies and keep the transition initial intensity scales, we used this transition as reference to normalize other transitions. Also, to follow the manual analysis procedures, when it was not the highest intensity, we preferentially defined its statistical features as 0 even if it contained intensities. To convert these features to be recognized by ML algorithm, we used the equations set out in Table 1 to calculate given features. To check our features' rationale, we visualized the MS raw data and connected them to the feature table. (FIG. 7, Table 2) We found that our designed features offered guidance for label separation. However, due to the number of features and difficulty associated with identifying samples, ML algorithms are useful for classifications.

Table 1. Features Description. A list of features classes derived from chromatography data. m: Median in the retention time; n: Number of retention timepoints between internal standard peak boundaries; Retention Time: {R1, R2, . . . Rm, . . . , Rn-1, Rn}. Target Coordinate: {(R1, T1), (R2, T2), . . . (Rm, Tm), (Rn-1, Tn-1), (Rn, Tn)}; Internal Standard Coordinate: {(R1, S1), (R2, S2), . . . (Rm, Sm), (Rn-1, Sn-1), (Rn, Sn)}; Max(T): Maximum value in the {T1, T2, . . . , Tn} set; Norm (T): Ti−Tmin/Tmax−Tmin in the {T1, T2, . . . , Tn} set which Tmax is the maximum intensity in the set and Tmin is the minimum intensity in the set; Corr(T,S):Σ(Ti−T)(Si−S)I\sqrt{Σ(Ti−T)Σ(Si−S)}; First (D): The first value in the {D1, D2, . . . , Dn} set; Last (D): The last value in the {D1, D2, . . . , Dn} set.

| Category | Feature | Description | Calculation |
|---|---|---|---|
| Statistical | PeakMax | The target maximum intensity between | Max {T1, T2, . . . , Tn-1, Tn} |
| | | the internal standard peak boundaries. | |
| | PeakArea | Summation of all target intensities between the internal standard peak boundaries | ${\sum }_{i = 1}^{n} Tn$ |
| | PeakRatio | Among the internal standard peak | Σᵢ₌₁ⁿTn/Σᵢ₌₁ⁿSn |
| | | boundaries, ratio of summation of target | |
| | | intensities to summation of internal | |
| | | standard intensities | |
| | Peak_Shift | The retention time difference between | Rm-R(Tmax) |
| | | internal standard and target intensities | |
| Morphological | Updown-Dif | The shift in target peak is defined as | \|Norm(Tmax) − Norm(Smax)\| |
| | | difference intensities between the left | |
| | | and right peak boundaries | |
| | Similarity | Pearson correlation coefficient between | Corr({T1, T2, . . . Tn}, |
| | | the internal standard and target | {S1, S2, . . . Sn}) |
| | | intensities | |
| | Symmetry | Pearson correlation coefficient between | Corr({T1, T2, . . . , Tmax}, |
| | | the left and right halves of the peak | {Tn, Tn-1, . . . , Tmax}) |
| | | intensity vector divided at the timepoint | |
| | | in the middle. | |
| | Jaggedness | The jaggedness score is defined as the difference of mean intensities at the same timepoint between standard and target peaks. | $\frac{1}{n} {\sum }_{i = 1}^{n} ( Norm ( T ) - Norm ( S ) )$ |
| | Modality | The modality score is defined as the | Di = Norm(Ti) − Norm(Si) |
| | | largest unexpected dip in the peak, | DPOS = { . . . , Di, . . . }, (Di >= 0) |
| | | normalized by peak height | \|First(DPOS) − LAST(DPOS)\| |
| FWHM | | The retention time scans between two | Rlef = R(Tmax/2), (R < Rm) |
| | | half-maximum intensities | Rright = R(Tmax/2), (R > Rm) |
| | | | \|Rleft − Rright\| |

TABLE 2
Features of two samples in FIG. 7.

| Category | Feature | MLIT0003 | MLIT0005 |
|---|---|---|---|
| Statistical | PeakRatio | 0.092821 | 0.010225 |
| | PeakMax | 1451.042 | 331.1061 |
| | PeakArea | 9550.851 | 3355.502 |
| | PeakShift | 2 | 2 |
| Morphological | Updown-dif | 0.111061 | 0.016681 |
| | Similarity | 0.923153 | 0.0348625 |
| | Symmetry | 0.98394 | 0.966846 |
| | Jaggedness | 0.148957 | 0.306734 |
| | Modality | 0.303865 | 0.751799 |
| | FWHM | 5 | 9 |

Prediction Performance of Machine Learning Models.

To automate data processing for targeted MS assays and accelerate their clinical application for TB diagnosis, we developed a ML model for automated MS data analysis using 80 features identified and quantified from raw data files. In the training dataset, each sample was labeled as ‘positive’ or ‘negative’ for serum CFP10pep signal based on a combination of empirical expertise and several quantitative measurements, such as the signal-to-noise ratio derived by commercial software. From the former results, although both statistical and morphological features had described the target peptide from different angles, it is difficult to use them to distinguish the difference among sample labels manually. To deal with given features efficiently, ML algorithms were introduced to assist with classification. To find the best way to make predictions, we incorporated multiple algorithms into analysis workflow instead of a single method. Due to the issues handling binary labels, we used logistic regression (LR), random forest (RF), and gradient boosting (GB) algorithms. These algorithms could use fewer samples to build models than other artificial networks which required huge training datasets. To compare if our novel engineered features could improve predictions, we followed a one-dimension traditional feature extraction and conversion pipeline then inputted these features to later performance analysis in parallel.

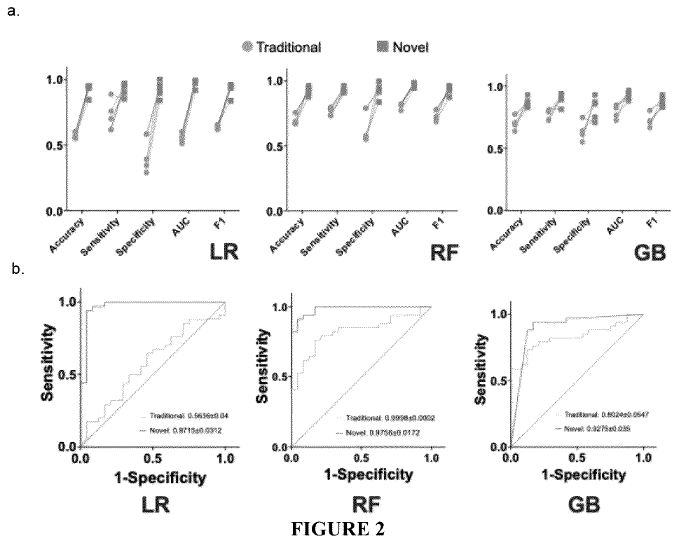

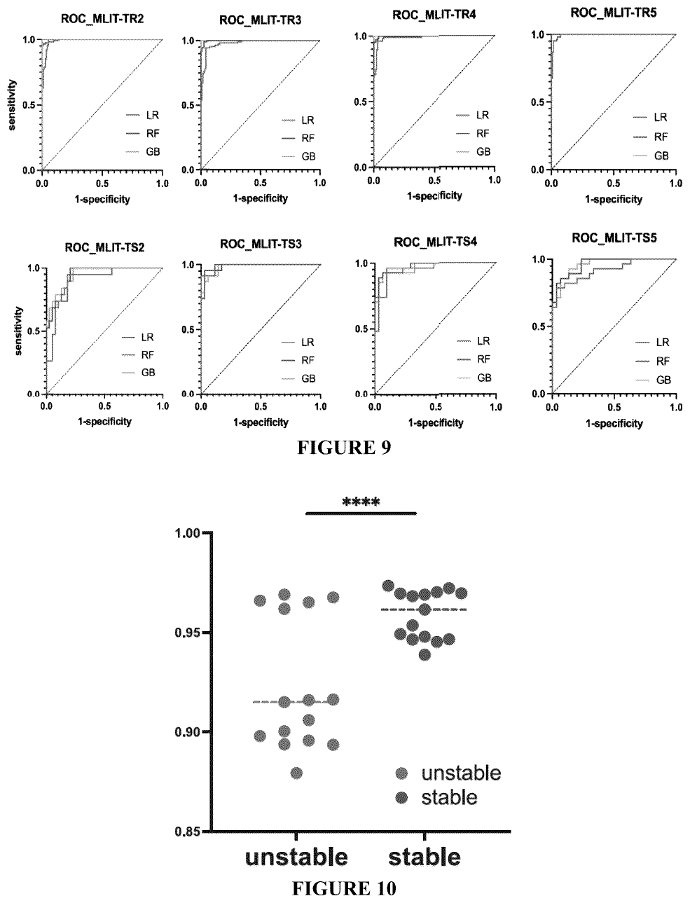

To evaluate if the algorithms could classify the samples by given features, we separate 80% of the MLIT dataset as a training dataset and the remaining 20% as a testing dataset. When the models were built and applied into both training and testing datasets, five metrics, including accuracy, sensitivity, specificity, F1 score, and AUC values were used to evaluate their performance. (FIG. 2A.) To dismiss the probability of high performance by chance, we did this workflow by five random sampling instances. From evaluation of training dataset, the AUCs of our engineered features were significantly higher than the traditional features in LR algorithm, but there was no significant difference in the RF and GB algorithms (FIG. 8, Table 3). The reasons for low performance in LR may be related to overfitting. From the testing dataset, performance of our novel engineered features in three algorithms were significantly higher than models built by traditional features. (FIG. 2A) These results proved that the predictions were dependent on the quality of features, and ours have the capacity to establish the profile of given data by one-dimension data. According to previous research, AUC values, which are derived from sensitivity and specificity, are more representative of predictive capabilities than accuracy in evaluating ML algorithms. To visualize the diagnostic ability of a binary classifier system performance, receiver operating characteristic (ROC) curves were established (FIG. 2B.) and the AUC values also were calculated by the area under the curve. To compare the model performance conveniently, the following statistical comparisons are made based on AUC values for dynamic active learning analysis. From the training dataset, the models by LR, RF, and GB three algorithms performed 0.7525±0.0302, 0.9988±0.0009, 1.0000±0.0000 which built on novel features and 0.9991±0.0008, 0.9998±0.0002, 1.0000±0.0000 on traditional features (errors calculated by 5 random re-samplings of training samples) AUC values separately. (FIG. 9) Meanwhile, using these best performing models applied to corresponding testing datasets, from the ROC curves, we saw the novel features constructed better classifiers in three algorithms (LR: 0.9715±0.0312; RF: 0.9756±0.0172; GB: 0.9275±0.035) than traditional features (LR: 0.5636±0.04; RF: 0.8123±0.0215; GB: 0.8024±0.0547). (FIG. 2B) Taken together, novel features which added morphological traits and dismissed certain redundancy information supported the high predictions efficiently.

TABLE 3
AUC values comparison among models built by traditional
and novel features. AUC values displayed as (mean ± SD).

| | Features | |
|---|---|---|
| Algorithm | Traditional | Novel |
| LR | 0.7525 ± 0.0302 | 0.9991 ± 0.0008 |
| RF | 0.9988 ± 0.0008 | 0.9998 ± 0.0002 |
| GB | 1 ± 0 | 1 ± 0 |

Comparison of Algorithms on Different Training Sample Sizes.

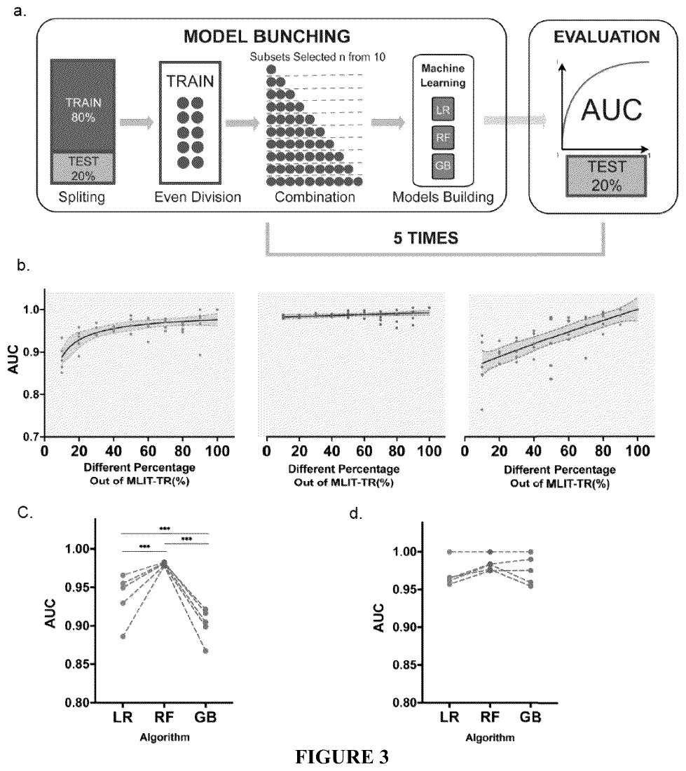

To address issues of limited databases and variance among different studies, it is beneficial to have a ML pipeline for use with datasets containing a relatively small sample sizes. To validate the performance of models built by small training datasets, we designed different volumes of training samples. (FIG. 3A.) To observe the performance trend, we compared the results by increasing training samples. We defined the training datasets out of the MLIT-TRn (n means the orders of resampling training dataset) dataset, and the subsets were created by randomly selecting 10-100% (incrementing by 10%) of the MLIT-TR to train the algorithms. The 10 subsets were named MLIT-TRn-1,MLIT-TRn-2, through MLIT-TRn-10. Also, we used the MLIT-TEn as the testing dataset to compare the models' performance. The whole procedure was called ‘model bunching’ as further set forth in the following section. From the result, we found that the model performance would keep increasing and achieve a relatively stable status. (FIG. 3B.) To further compare the models from the changing performance, we separated them into two phases, ‘unstable’ (built with 10-50% of training data, AUC value: 0.9397±0.0463) and ‘stable’ (built with 60-100% of the training data, AUC value: 0.9692±0.0269) through statistical analysis (FIG. 10). When we analyzed each algorithm's performance in two phases, logistic regression and gradient boosting algorithms had significant differences. (FIG. 12) However, the random forest algorithm maintained performance consistently.

To compare the performance among the algorithms, the pairwise t-test method was used at both unstable and stable stages. In the unstable stage, the random forest algorithm (0.9802±0.0045 had statistically higher AUC values than logistic regression (0.9372±0.0345) and gradient boosting (0.9017±0.0465). (FIG. 3C.) In the stable stage, the random forest algorithm maintained a statistically higher AUC (0.98±0.014) than linear regression (0.9641±0.0221), although there was no longer a statistical difference compared to gradient boosting (0.9702±0.0255). (FIG. 3D.) From these results, a random forest algorithm could assist given features to enforce better predictions even there were limited samples in the training dataset.

External Validation

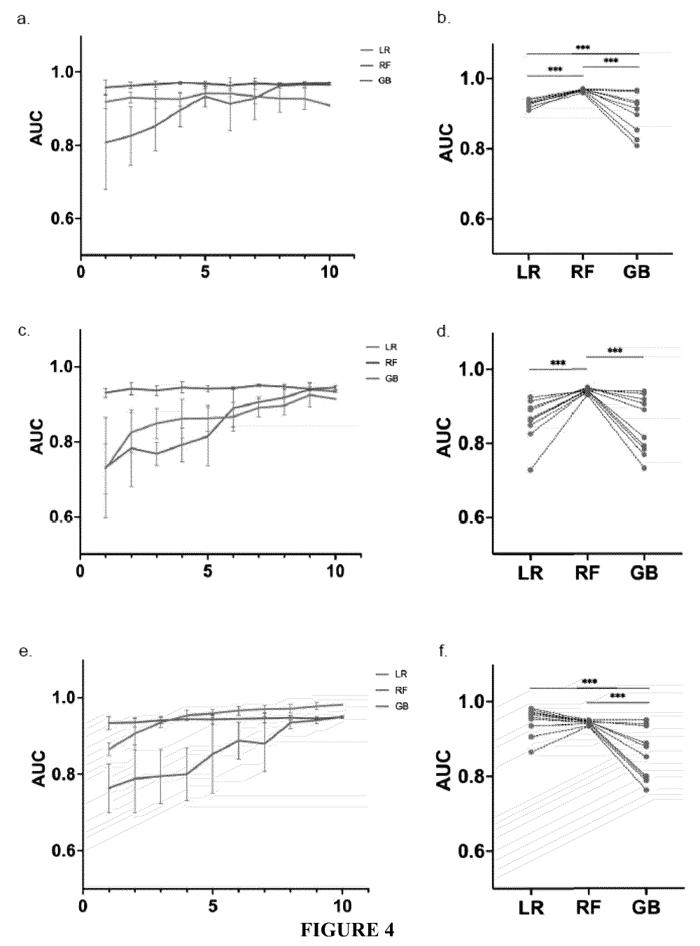

To prove the generalizability of internal models, we used unbalanced and low-quality data to validate the models. There were three independent datasets available for external validation, INDTS1, INDTS2, and INDTS3. The INDTS1 dataset contains 201 samples with 13.93% of samples labeled as biomarker positive. Also, most samples were collected from children who were infected by HIV and TB together, so we are not sure if the interaction would have influence on biomarker identification by machine learning methods. Linear regression, random forest, and gradient boosting algorithms were tested using the same “model bunching” procedures described above. To compare model performance, AUC values were calculated and compared to display their difference. (FIG. 4A.) The random forest algorithm maintained high performance (AUC: 0.9666±0.0084) throughout the entire 10-100% range of data training, with additional data points having minimal improvements on model performance. Logistic regression (AUC: 0.928±0.0212) and gradient boosting (AUC: 0.905±0.0793) displayed changing performance and both were lower than random forest. To test which was the best algorithm to build pipeline out of the three, we did statistical analysis to compare their performance and the result showed that the random forest had significantly higher AUC than the other two algorithms. (FIG. 4B.) This consistent, high performance of the random forest algorithm on the INDTS1 dataset independently confirms the robustness of its predictive power. Logistic regression and gradient boosting both had significantly lower AUC values compared to the random forest no matter what percentage of the training samples was used.

INDTS2 contains 36 samples, with 44.44% of samples labeled as biomarker positive. Samples were collected 10 years ago, and long-term storage and multiple transformations likely contributed to the degraded quality of the samples. After the “model bunching” procedures, we saw the changing of performance followed a similar pattern to MLIT's (FIG. 4C.). To test if the best algorithm was still random forest, we did statistical analysis to compare their performance. Then, the random forest (AUC: 0. 942±0.0104) provided significantly higher predictions than both logistic regression (AUC: 0.8621±0.0648) and gradient boosting (AUC: 0.8477±0.0941) (FIG. 4D.) It displayed the same conclusion as previously, and our pipeline could detect the samples even though their qualities were low.

In INDTS3, there were 98 samples, and the positive sample rate was 19.38%.

Samples of this study were collected by a TB prospective cohort project which the biomarker results closed to patients' clinical diagnosis. The difference from previous studies was that the background of MS data in this cohort was relatively higher. This reason may have effects on data processing in which higher noise misled the models to make false predictions. When we applied the ‘model bunching’ again in this cohort, the performance by random forest was consistent as usual and gradient boosting had higher variance than other studies. But the performance of logistic regression increased during the accumulating samples into training dataset, even beyond random forest with 40% of training samples. (FIG. 4E.) Further, its performance (AUC: 0.9486±0.0387) had significantly higher than random forest (AUC: 0.9429±0.0099) and gradient boosting (AUC: 0.8589±0.0879). (FIG. 4F.) These results illustrated that the best algorithm would be dependent on the data input. In other words, ML models also needed to be optimized by external datasets instead of static pipelines.

Taken together, even though the samples and collection procedures were different from the internal dataset, our workflow also provided accurate predictions for external datasets. Additionally, the performances of logistic regression and gradient boosting increased with training sample amplification. In contrast, the random forest algorithm offered the best classification ability by given features steadily and consistently both at internal and external validations. Although the random forest algorithm did not provide the best prediction in any dataset, it could afford limited training samples and stable performance in unknown datasets. Then, we would do more analysis on the method based on our features and random forest algorithm.

Dynamic Active Learning Workflow

Above, the MLIT dataset was used for both internal and external validation. Because the positive labeled sample ratio was 55%, the training datasets were relatively label-balanced by simple random sampling methods. Increasing training datasets also provided more accurate results because they gained diverse samples for classification. The ideal dataset for machine learning pipeline construction should be diverse and label balanced because such dataset could assist the model to cover all characteristics to make unbiased predictions. However, it is common to not have enough samples at the initial period, and the coming samples could not ensure they were of ideal status. Also, it is common to receive batches of samples containing one property instead of both balanced labels. Then unbalanced labeled training datasets would have effects on the model performance. So mechanisms for training sample optimization are desired. From the concepts of the active learning approach, optimal training samples were filtered by multiple procedures out of given samples or dynamic increasing samples to realize better predictions on unknowns.

To deal with these situations, we built our own active learning (AL) method for dynamic training dataset construction. To mimic the balanced and unbalanced labeled training datasets, we selected positive and negative samples out of MLIT cohort studies with different positive sample rates randomly as components of dynamic training datasets. To initialize the AL pipeline, we picked 5 positive and 5 negative samples as the initial dataset. To compare the performance by AL method, we use the passive learning (PL) method, which means input all incoming samples without consideration of their labels and features. To show the variance of different dynamic dataset selections, we did sampling by 3 times with different pseudo-random numbers. From the left lane of results (FIG. 6C, a), the AUC values of the AL method were more stable and higher when the training datasets contained unbalanced labeled samples.

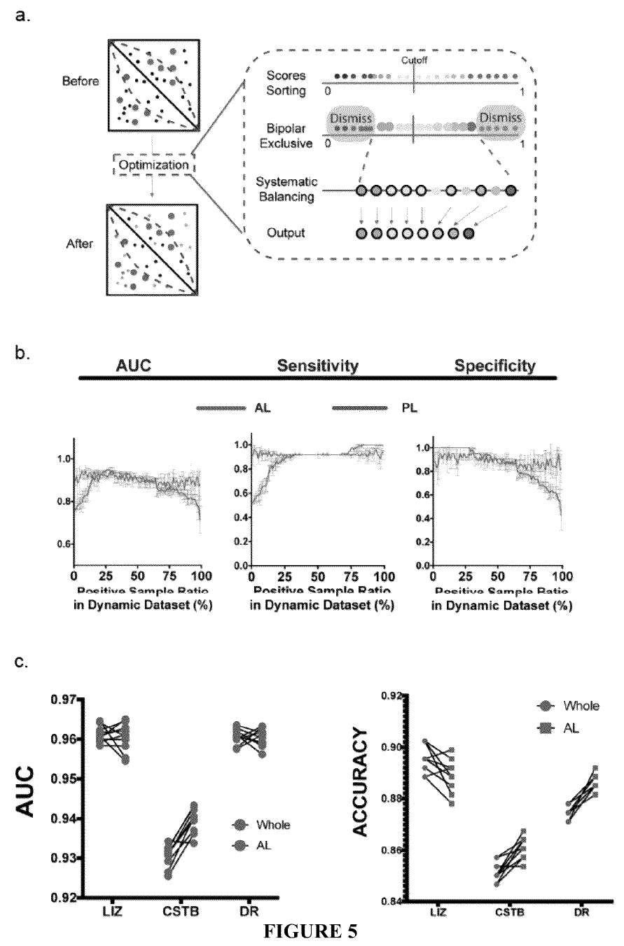

For different purposes, certain researchers or physicians concentrate on sensitivity or specificity of model performance in the clinical field. To analyze if AL improved them individually, we compared the methods on sensitivity and specificity separately. We saw the sensitivity and specificity (FIG. 5A, middle and right lanes) had a high relationship to the training dataset components of binary labels by the PL method, but the AL method provided stable and close to perfect predictions no matter what positive label rate was in the dynamic dataset.

For internal validation, samples of the testing dataset contained similar characteristics to the training dataset. To test if the AL method improved external dataset prediction, we replaced the three external datasets to internal testing dataset with the same AL pipeline. From the AUC values (FIG. 5B left lane.), even the PL method was not affected by the unbalanced training dataset than internal validation, the AL method kept higher than PL method. Furthermore, the sensitivity of three external validations (FIG. 5B middle lane), it consumed more positive samples to achieve high performance of models in the PL method, but the AL method established stable sensitivity no matter what percentage of the positive samples in the dynamic datasets. Also, the same situation applied to specificity (FIG. 5B right lane.), when the AL method held certain predicting levels even though the negative decreased in the dynamic dataset. Meanwhile, we could see the AL method have higher variance when the dynamic contained one property of samples, the AL method sacrificed certain samples to ensure unbiased predictions for next unknown samples. Although there were heavy vibrations at two extreme instances of positive and negative percentages in the dynamic datasets, AL still provided better classifications comprehensively.

We concluded that the AL method not only provided high performance in predictions, but also satisfied different objectives on specific studies. Meanwhile, this pipeline grows and optimizes by itself instead of manual operations on static training iteration.

Interpretable Feature Importance for Machine Learning Model.

Machine learning models should not only have high classification ability but also provide insight into the importance of features for predictive power. Machine learning methods, especially tree-based models, have previously been regarded as ‘black boxes’. However, in research and in the clinical field, results need to be explained and rationalized for any method or model. To understand how machine learning works on the given features, we combined several workflows to explain why ML methods displayed high performance in classification. In recent years, SHAP values have been used as an essential quantification to display the contribution of each individual feature in tree-based models, and show the rankings of feature importance by the dispersion of SHAP values (the higher the dispersion, the more important for final prediction).

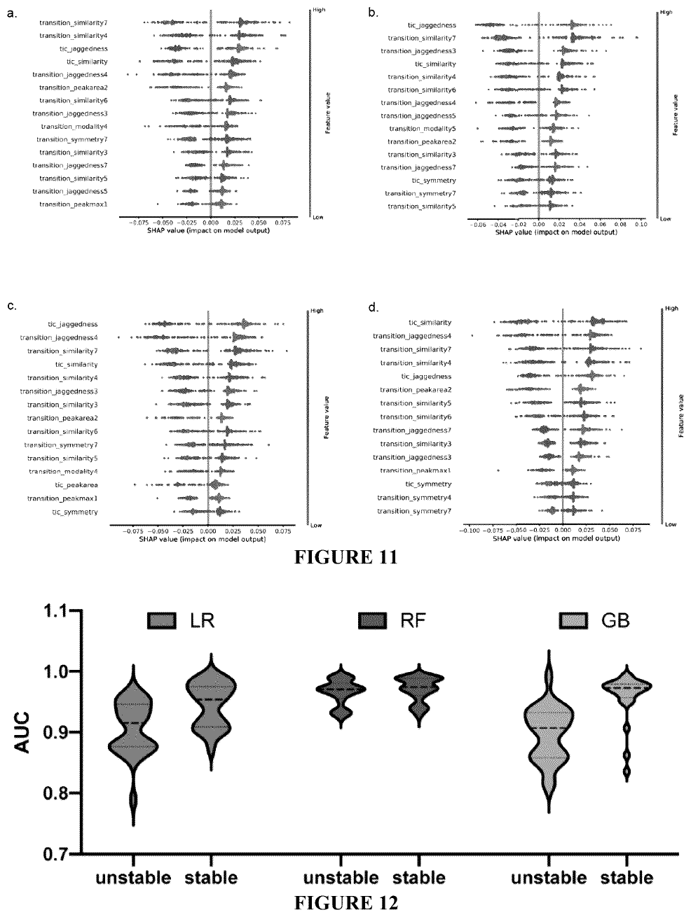

The random forest algorithm could enable given features to offer stable and high performance not only in limited training samples but also in external validation. So, we choose it as the main algorithm to analyze the MS data. To explore why the ML models could give and sustain perfect predictions, we used the model trained by MLIT-TR1 dataset as a demo to display their inner structures. To visualize feature importance for the random forest algorithm, the mosaic plot was used to display SHAP value rankings (top 15 displayed), feature categories, and feature classes. The scatterplot displayed the distributions of original SHAP values by features. Dots' color displayed the samples' label (Red=‘positive’; Blue=‘negative’). Also, the feature importance was sorted by the mean of total absolute SHAP values. (FIG. 5A.) To test if the rankings were repeatable, scatterplots of models built by MLIT-TR 2-5 were also generated. (FIG. 11) From these results, we found that the models depended on morphological features more than statistical ones in predictions, and the features extracted from larger transitions tended to help more than smaller ones. To confirm these assumptions, we did a statistical analysis and established more evidence.

In manual analysis, expertise preferred to observe the chromatography and combined parameters which helped to extract signals out of backgrounds. In contrast, the original intensity was secondary when the samples contained obvious peak signals even if their intensities were low. To understand if ML methods followed this trait, we compared morphological feature importance to statistical by SHAP value ranking. (FIG. 5B.) Through the nonparametric rank-sum test, we found that morphological features have higher importance than statistical features. Due to unstable sampling in random forest algorithm, SHAP value rankings of 5 models trained by different re-sampling datasets illustrated the pattern was a subtle difference, but not significantly.

In summary, SHAP values help to rank feature contributions to categorization and provide the rationale behind machine learning algorithm decision-making. Morphological features mimic human observation and have greater impacts on model performance through nonparametric comparisons than statistical features described in original data. Additionally, individual transitions from MS2 helped to elaborate samples at both categories. Furthermore, higher m/z transition intensities contributed more to the predictive power of the algorithm when compared to lower m/z intensities. A ML method also could detect it and avoid being misclassified automatically. Although no weights or biases were given to the algorithms, the machine learning methods could discern certain rules to drive reasonable and acceptable decision-making as manual operations.

### DISCUSSION

LC-MS/MS data has specific characteristics with its two-dimensional data structure, i.e. retention time and m/z. Combining these two signals is a challenge, and discerning relationships between the two signals would rely on high-dimension, complex algorithms. Instead, generating mass spectrometry data features can remove the need for higher-dimension algorithms, and can potentially reduce the computational intensity of LC-MS/MS data analysis in real-world applications.

Thus, managing and designing efficient LC-MS/MS data features was a critical step in building the machine learning pipeline. In the present study, both target peptide and internal standard peptide features were generated and utilized, including similarity, jaggedness, and the ratio between the target and internal standard intensities. These features were utilized to aid the algorithm in understanding the relationship between target and internal standard intensities and help to improve the predictive capabilities of the models.

Additionally, the entirety of chromatography data was retained and utilized in feature extraction. In traditional analysis of peptide intensity, commercial software often ignores signals in MS1 or MS2 when intensities are below certain thresholds. However, these signals can be detected and used by ML algorithms. In this study, these low intensity signals were captured and used as key gradients in peptide classification.

Furthermore, in traditional analysis, it is difficult to separate fragments generated from MS2 from those generated from MS1. Machine learning methods can recognize the interactions among the various fragments, and determine which step certain transition intensities were generated from by comparing to other samples at the same m/z. Different transition intensities within different features have specific traits, which can be utilized by algorithms to more accurately classify when a fragment was generated.

The previously described steps of data analysis and feature generation were used to train the machine learning algorithms in differentiating positive from negative samples, and to identify differences that are not typically detected or utilized by traditional analysis.

Generating Machine Learning Pipeline.

Supervised machine learning utilizes features and labels in datasets to train algorithms. Three machine learning models (logistic regression, random forest, and gradient boosting) were selected based on their characteristics with regards to binary outcome predictions. Logistic regression models use maximum likelihood to adjust the coefficient of input features to improve classification ability and achieve the lowest error. Random forest models utilize out-of-bag methods to make predictions through decision trees, using feature entropy to classify binary outcomes. Gradient boosting uses weak learners to make predictions, and adds additional weak learners to optimize a loss function. Each algorithm has pros and cons, and therefore, each has different use cases for outcome prediction.

In the present disclosure, after hyperparameter adjustments and generation of features from LC-MS/MS data, all three supervised machine learning algorithms had strong performance in classifying positive and negative samples in both the training and testing datasets.

Additionally, a K-Fold cross validation method was utilized to decrease the risk of over-fitting. Although there were slight differences in model performance on the training dataset compared to on the testing dataset, the difference was not statistically significant.

Impact of Sample Size on Model Performance.

A larger training dataset often results in more robust machine learning algorithms. However, there are often obstacles in obtaining large-scale datasets at the onset of a study or novel diagnosis method, including limit case count and the time-intensive nature of manually analyzing LC-MS/MS data. Thus, developing machine learning algorithms capable of high and stable performance even with limited training data is a challenge in implementing ML models in real-world practice.

In the present disclosure, the training dataset was divided into 10 subsets (by randomly selecting 10-100% of the data, incrementing by 10%) to train the algorithms and compare model performance based on the size of the training data. The random forest algorithm had the most stable performance during both the unstable (10-50% of training data) and stable (60-100% of training data) stages. Even when training data points were limited (i.e. in the unstable training stage), the random forest algorithm was able to maintain consistent, accurate performance in classifying LC-MS/MS data. This may be due to the characteristic of random forest algorithms in selecting the main distinguishable features from data, rather than considering all features in totality.

Extrapolation of Machine Learning Models.

In previous studies, results from machine learning models were difficult to replicate utilizing external datasets, often due to the extraction of crude features which inputted LC-MS/MS data directly. To ensure that future ML models are accurate and extrapolate, it has been suggested that a wide range of populations and studies be utilized during ML training, for example by using representative population-level databases or incorporating several datasets focused on different disease and biomarker studies. However, these solutions would be difficult and time-prohibitive to implement, and researchers and clinicians may simply opt to continue manual analysis of LC-MS/MS data instead.

The present disclosure addresses this challenge by designing features from LC-MS/MS data for use in the ML models (rather than input LC-MS/MS data directly into models). This resulted in consistent performance on not only the testing dataset, but also on two external independent datasets. The random forest algorithm maintained high and stable performance throughout all stages in all three datasets utilized. Of note, the differences among the three machine learning algorithms seemed to be magnified in testing on independent datasets compared to testing on the MLIT-TS datasets. A potential area of additional research is understanding whether data points from these independent datasets and the magnified differences observed could be used to further train the algorithms and improve classification abilities.

SHAP Values.

Regardless of the predictive power of a model, it is desirable to have comprehensive rationale behind how algorithms derive their predictions. Especially in biological and medical fields, researchers and physicians require accurate quantification of measurements and strong supporting evidence. There are several ways to analyze feature importance in machine learning models.

In logistic regression algorithms, feature importance can be evaluated by the statistical significance of each feature parameter. However, this may become less accurate in large-scale datasets as the number of features increase. Another method is permutation of feature importance, where prediction errors are calculated from various models utilizing a subset of the total features. However, this process can become time prohibitive in large datasets with numerous features.

SHAP values have increasingly become a popular and acceptable indicator of feature importance, and have the capacity to quantify feature importance in random forest algorithms.

In the present disclosure, each feature's importance was ranked by its SHAP value, and this ranking allowed for a visualization of feature contribution to the model as a whole. SHAP value rankings also made intuitive sense and were aligned with the philosophy behind traditional analysis. For example, morphological features (i.e. features based on the original data) were observed to have greater impacts on model performance through nonparametric comparisons than statistical features. In traditional analysis, morphological features are more recognizable and therefore more regularly used in categorization. Additionally, higher m/z transition intensities contributed more to the predictive power of the algorithm when compared to lower m/z intensities. This may be due to lower m/z transitions being harder to differentiate, and lower transition intensities being more likely to be miscategorized as noise during signal processing.

Although no weights or biases were given to the algorithms, the machine learning methods could automatically discern certain rules to drive decision making. Both SHAP value rankings and the intuitive reasoning behind model decision making offer strong rationale for the categorical abilities of the ML algorithm presented in this paper.

Dynamic Optimization Workflow

In practical application, we usually faced the following issues: 1). Initial sample size were limited, and the big dataset was constructed gradually; 2). The data quality would be different from batch to batch, a static training dataset could not adapt to the new samples immediately. Machine learning pipeline should contain a mechanism to get new samples involved automatically. 3). New samples from certain batch could be labeled-bias samples which the samples only contain one label or property, the prediction would be affected if these samples were injected into training dataset. Hence, our dynamic machine learning pipeline was not only solve these issues, but also provide an idea that we could design rules to let the model optimize dynamically instead of adjusting parameters manually. Moreover, by conventional method, people would consider about the data quality or set some thresholds of important features. On the contrary, our approach used the scores which from the model-calculation to decide if new samples were suitable as training samples, and we didn't need to spend time on the key factor identification or data quality filtering.

In certain embodiments, the present invention utilizes a model bunching method for detection of the limitations of training sample size and the development of small datasets for ML optimization with the capacity to adapt a new study or population quickly and make predictions using few initial samples. In certain embodiments, the present invention allows for the utilization of morphological MS features to enable ML models to be built lightly and efficiently. In certain embodiments, the present invention allows for the extension of MS data to multiple labeled MS data.

In summary, by using suitable features generated from LC-MS/MS data and dynamic machine learning pipelines, this study constructed a powerful ML classifier with high performance. The random forest algorithm displayed not only highly predictive power, but also stable performance even with limited training data. The model's performance was replicable across two independent datasets. Furthermore, this project explicitly compares feature importance, enabling a more comprehensive understanding of the ML model's classification methodology. These features and procedures offer a novel method to help with biomarker detection in certain disease studies.

The following references are incorporated by reference in their entirety for all purposes.

What is claimed is:

1. A method of determining presence of an analyte in a sample, comprising the steps of:
a) obtaining mass spectrometry (MS) data from a sample;
b) extracting, by a computer, features from the MS data;
c) inputting, by a computer, the features extracted in step b) into a trained prediction model, wherein the prediction model is trained to predict presence of an analyte in said sample; and
d) generating an output, wherein the output comprises prediction of the presence of the analyte in said sample.

2. The method of claim 1, wherein the features comprise statistical features and morphological features.

3. The method of claim 2, wherein the statistical features comprise Peak_Max, Peak_Area, Peak_Ratio, and/or Peak_Shift.

4. The method of claim 2, wherein the morphological features comprise: updown-difference, similarity, jaggedness, modality, symmetry, and/or FWHM.

5. The method of claim 4, wherein the morphological features are extracted using normalized MS data.

6. The method of claim 1, wherein in step d) the output further comprises feature importance.

7. The method of claim 6, wherein the feature importance is obtained by calculating a Shapley Additive exPlanation (SHAP) value for each extracted feature, and sorting the features by the SHAP value.

8. A method of building a machine learning pipeline, comprising the steps of:
a) extracting features from mass spectrometry (MS) or liquid-chromatography mass spectrometry (LC-MS) data regarding presence of an analyte;
b) constructing, by one or more computing devices that implement a machine learning program, two or more machine learning models using an active learning workflow;
c) optimizing, by the one or more computing devices, the machine learning model; and
d) selecting, by the one or more computing devices, a best model;
wherein the features in step a) comprises statistical and morphological features.

9. The method of claim 8, wherein the active learning workflow comprises at least one of:
(i) label balancing, and (ii) even score distribution.

10. The method of claim 9, wherein the label balancing comprises randomly providing positive rate of training dataset.

11. The method of claim 9, wherein the even score distribution evaluates at least one of the following: accuracy, sensitivity, specificity, area under curve (AUC), and F1.

12. The method of claim 8, wherein the features comprise statistical features and morphological features, and wherein the morphological features are extracted using normalized MS data.

13. The method of claim 8, wherein the machine learning model in step c) comprises training set optimization.

14. A system, comprising:
a) at least one processor;
b) a memory, storing program instructions that when executed by the at least one processor causes the at least one processor to perform a machine learning pipeline, the machine learning pipeline is configured to perform at least one of the following modes:
i) training mode:
(A) receive mass spectrometry data of a sample;
(B) extract at least one feature from the mass spectrometry data, wherein the at least one feature is a statistical feature and/or a morphological feature;
(C) optimize training dataset by active learning strategy; and
(D) select a best prediction model;
ii) prediction mode:
(A) receive mass spectrometry data of a sample;
(B) extract at least one feature from the mass spectrometry data, wherein the at least one feature is a statistical feature and/or a morphological feature; and
(C) generate an output of determining whether an analyte is present in the sample.

15. The system of claim 14, wherein the at least one feature comprises statistical features and/or morphological features.

16. The system of claim 15, wherein the statistical features comprise Peak_Max, Peak_Area, Peak_Ratio, and/or Peak_Shift.

17. The system of claim 15, wherein the morphological features comprise: updown-difference, similarity, jaggedness, modality, symmetry, and/or FWHM.

18. The system of claim 17, wherein the morphological features are extracted using normalized MS data.

19. The system of claim 14, wherein in step (C) of the prediction mode the output further comprises feature importance of the analyte and/or the status of the analyte.

20. The system of claim 19, wherein the feature importance is obtained by calculating a Shapley Additive exPlanation (SHAP) value for each extracted feature, and sorting the features by the SHAP value.

21. One or more non-transitory, computer-readable storage media, storing program instructions that when executed on or across one or more computing devices cause the one or more computing devices to implement at least one of:
a) training mode:
i) receive mass spectrometry data of a sample;
ii) extract at least one feature from the mass spectrometry data, wherein the at least one feature is a statistical feature and/or a morphological feature;
iii) train the machine learning pipeline by optimizing a training model; and
iv) select a best prediction model;
b) prediction mode:
i) receive mass spectrometry data of a sample;
ii) extract at least one feature from the mass spectrometry data, wherein the at least one feature is a statistical feature and/or a morphological feature; and
iii) generate an output of determining whether an analyte is present in the sample.

22. The non-transitory, computer-readable storage media of claim 21, wherein the feature comprises statistical features and/or morphological features, wherein the statistical features comprise Peak_Max, Peak_Area, Peak_Ratio, and/or Peak_Shift, and wherein the morphological features comprise: updown-difference, similarity, jaggedness, modality, symmetry, and/or FWHM.
